(19) United States
(12) Patent Application Publication
(10) Pub. No.: US 2022/0062348 A1
(43) Pub. Date: Mar. 3, 2022

(54) ISOLATED SEPTAL CARTILAGE EXOSOME USED FOR GENERATING CARTILAGE TISSUE

(71) Applicant: YEDITEPE UNIVERSITESI, ISTANBUL (TR)

(72) Inventors: Fikrettin SAHIN, Istanbul (TR); Pakize Neslihan TASLI, Istanbul (TR); Ezgi AVSAR APDIK, Istanbul (TR); Ayse Sezim SAFAK, Istanbul (TR)

(73) Assignee: YEDITEPE UNIVERSITESI, ISTANBUL (TR)

(21) Appl. No.: 17/277,734

(22) PCT Filed: Sep. 17, 2019
(86) PCT No.: PCT/TR2019/050764

§ 371 Date: Mar. 19, 2021

(30) Foreign Application Priority Data
Sep. 19, 2018 (TR) 2018/13459

(51) Int. Cl. A61K 35/32; C12N 5/077

(52) U.S. Cl. CPC A61K 35/32; C12N 5/0655

(57) ABSTRACT

A cartilage is generated by a formulation, wherein the formulation is produced by an isolated septal cartilage exosome released by cells isolated from a septal cartilage to a medium. The objective of the isolated septal cartilage exosome is to generate the cartilage to be used in a treatment of cartilage tissue defects, such as osteoarthritis or arthrosis, since the cartilage induces a cartilage formation and also suppresses an inflammatory response.

### CROSS REFERENCE TO THE RELATED APPLICATIONS

This application is the national stage entry of International Application No. PCT/TR2019/050764, filed on Sep. 17, 2019, which is based upon and claims priority to Turkish Patent Application No. 2018/13459, filed on Sep. 19, 2018, the entire contents of which are incorporated herein by reference.

### TECHNICAL FIELD

The present invention relates to a formulation which is used for generating cartilage and is produced by the microvesicles that are released by the cells isolated from septal cartilage to the medium.

### BACKGROUND

Cartilage is a flexible, hard and white tissue that performs the function of bone in some organs. In most of the primitive vertebrates and in developed vertebrates, the skeleton of the embryo is composed of cartilage. In a fully-grown human body, there are cartilage parts in the nose, larynx and ears. It also serves as a cushion covering the faces of the bones, which form the joint, facing each other. The articular cartilage can be damaged and eroded in various ways. This results in a degenerative joint disease called osteoarthritis or arthrosis [1]. Osteoarthritis is a noninflammatory, chronic and degenerative disease characterized by progressively occurring cartilage destruction, osteophyte formation, and subchondral sclerosis especially in the load-bearing joints. In this disease, which is also called degenerative arthritis, osteoarthrosis or hypertrophic arthritis, there is a gradual loss of joint cartilage [2].

Unlike the bone, the cartilage does not need to come into direct contact with the bone in order to be alive. When tissue fluids reach the fibrous matrix of the cartilage, the chondroblasts are nourished and, unlike the alloplastic implants, they do not have to be embedded in the tissues. Therefore, it can be easily used in the nasal ridge and even in the subepithelial pockets. For this purpose, ready-to-use cartilages or septal, conchal or costal cartilage can be used. The cartilage can be easily shaped and can be used both as a support and as a filling material for minor defects and edge irregularities in the nose due to its flexible structure. Most of the cartilage grafts that are used are autologous. Fresh or stored homologous cartilages and irradiated heterologous cartilages have been used for many years, but their use has been reduced as they are resorbed over time 131. Septal cartilage, avascular and rib cartilage are widely used in soft tissue defects in the head and neck region and to replace nasal reconstruction procedures.

Studies on tissue engineering have attempted to form cartilage in vitro and in vivo by seeding the suitable cells into the suitable resorbed biomaterial scaffolds. Furthermore, tissue engineering of human septal cartilage for soft tissue replacement in the head-neck region has the potential to provide clinical benefit in the near future [4].

In cartilage losses, self-healing capacity of the tissue is very limited. Although limited repair occurs, the resulting tissue is a fibrous cartilage, which does not have the same biomechanical properties with the original articular cartilage. Therefore, the aim of cartilage tissue engineering is that the obtained artificial cartilage has the same biomechanical properties with a normal articular cartilage [5]. In the clinical studies that are conducted, it is seen that the techniques used for cartilage repair provide short and medium term results. Extensive research is underway for second-generation tissue engineering solutions for cartilage repair. Various approaches and new techniques that will allow arthroscopic implantation of cells and thus reduce morbidity are being investigated. None of the numerous techniques available today is able to consistently reproduce normal hyaline cartilage, and the best long-term treatment is still unknown [6]. Biomechanical tests have proven that the biomechanical properties of tissue-engineered cartilage are compatible with those of normal septal cartilage [4, 7].

Current therapeutics against cartilage defects include surgical interventions (e.g., microfracture, mosaicplasty, tissue engineering including advanced and mimetic biomaterials scaffolds), cell transplantation (stem cell or chondrocyte implants), targeted therapy and disease modifying therapy (anti-inflammatory agents) [8].

The problems encountered in the state of the art can be listed as follows:
The inflammation and immune response that the materials; which are used in cartilage formation, tissue engineering, therapeutic studies and aesthetic cartilage transplantation, cartilage formation and cartilage filling; develop against the body and the cells restrict the use of these materials;
The activity of these materials on cartilage formation is inadequate;
In studies wherein cellular therapy is performed, the subsequent complications that will be caused by the cells are unknown;
The short-term effects of the treatments are inadequate in the long term.

European patent application document numbered EP2551342, one of the state of the art applications, discloses a method for inducing differentiation of human inferior turbinate mesenchymal stromal cells to cartilage cells, bone cells, nerve cells or fat cells. The method of the said invention is a method for isolating and culturing of cartilage cells.

European patent application document numbered EP3145514, one of the state of the art applications, discloses a formulation for regeneration of bone, cartilage, teeth, and periodontium. By administering the formulation developed in the said invention, bone and/or cartilage growth is/are stimulated for the treatment of bone fractures and cartilage damage. In an experimental study conducted for developing the invention, the stem cells isolated from dental pulp are cultured in petri dishes in DMEM culture medium.

European patent application document numbered EP1926507, one of the state of the art applications, discloses an implant for the repair of cartilage defects and a method for manufacturing the said implant. The implant comprises an implant body of natural cartilage tissue being coated with autologous cells having a chondrogenic potential. These cells are produced by in vitro cell proliferation starting from the chondrocytes isolated from a cartilage biopsy.

The United States patent application document numbered US2017296590, one of the state of the art applications, discloses a composition for inducing chondrocyte differentiation or regenerating cartilage tissue. The composition of the said invention includes exosomes derived from stem cells differentiating into chondrocytes. In the said invention, adipose stem cells are differentiated into chondrocytes and exosomes are isolated from the chondrocytes.

### SUMMARY

The objective of the present invention is to induce cartilage formation from the isolated septal cartilage exosomes for aesthetic and therapeutic purposes.

Another objective of the present invention is formation of a cartilage that does not generate immune response, inflammation, toxicity and irritation to the body and the cells thanks to its anti-inflammatory properties.

A further objective of the present invention is to obtain a cartilage tissue used in the treatment of cartilage tissue defects, such as osteoarthritis or arthrosis, from an isolated septal cartilage exosome since it induces cartilage formation and also suppresses the inflammatory response.

### BRIEF DESCRIPTION OF THE DRAWINGS

“Isolated septal cartilage exosome used for generating cartilage tissue” developed to fulfill the objectives of the present invention is illustrated in the accompanying figures, in which;

FIG. 1 is a graphical representation of the evaluation of the effect of administration of the exosomes obtained from septal cells to stem cells at different concentrations for 24, 48 and 72 hours on cell viability using MTS test.

FIG. 2A is a graphical representation of the evaluation of the effect exosomes obtained from septal cells and cartilage differentiation medium on CD44 gene expression levels by administering different concentrations thereof to stem cells.

FIG. 2B is a graphical representation of the evaluation of the effect exosomes obtained from septal cells and cartilage differentiation medium on SOX9 gene expression levels by administering different concentrations thereof to stem cells.

FIG. 3A is a graphical representation of the evaluation of the apoptotic effect of exosomes obtained from septal cells on cells by administration of only exosomes obtained from septal cells (100%) to stem cells.

FIG. 3B is a graphical representation of the evaluation of the apoptotic effect of exosomes obtained from septal cells on cells by administration of only exosomes obtained from septal cells (50%) to the stem cells.

FIG. 3C is a graphical representation of the evaluation of the apoptotic effect of exosomes obtained from septal cells on cells by administration of only exosomes obtained from septal cells (25%) to the stem cells.

FIG. 3D is a graphical representation of the evaluation of the apoptotic effect of exosomes obtained from septal cells on cells by only administering the cell medium to the stem cells.

FIG. 3E is a graphical representation of FIGS. 3A-3D.

FIG. 4A is a septal cell exosome.

FIG. 4B is an exosome/cartilage differentiation medium mixture (1:1).

FIG. 4C is a representation of Alcian Blue staining of the cells treated with cartilage differentiation medium for 10 days.

FIG. 4D is a control application of Alcian Blue staining of the cells treated with cartilage differentiation medium for 10 days by light microscope.

FIG. 5A shows the graphical representations of the effect of the Septal Cell exosome on Pollen Allergen-activated white blood cells in the scope of the present invention. (the graph of measurement of CD4 T helper lymphocytes, CD8 T cytotoxic lymphocytes, CD19 B lymphocytes and CD56 natural killer cells with the antibodies that are surface markers by flow cytometry device)

FIG. 5B shows the graphical representations of the effect of the Septal Cell exosome on Mite Allergen-activated white blood cells in the scope of the present invention. (the graph of measurement of CD4 T helper lymphocytes, CD8 T cytotoxic lymphocytes, CD19 B lymphocytes and CD56 natural killer cells with the antibodies that are surface markers by flow cytometry device).

FIG. 6A shows the graphical representations of the effect of the Septal Cell exosome on IL2 activated white blood cells in the scope of the present invention. (the graph of measurement of CD4 T helper lymphocytes, CD8 T cytotoxic lymphocytes, CD19 B lymphocytes and CD56 natural killer cells with the antibodies that are surface markers by flow cytometry device)

FIG. 6B shows the graphical representations of the effect of the Septal Cell exosome on PHA activated white blood cells in the scope of the present invention. (the graph of measurement of CD4 T helper lymphocytes, CD8 T cytotoxic lymphocytes, CD19 B lymphocytes and CD56 natural killer cells with the antibodies that are surface markers by flow cytometry device).

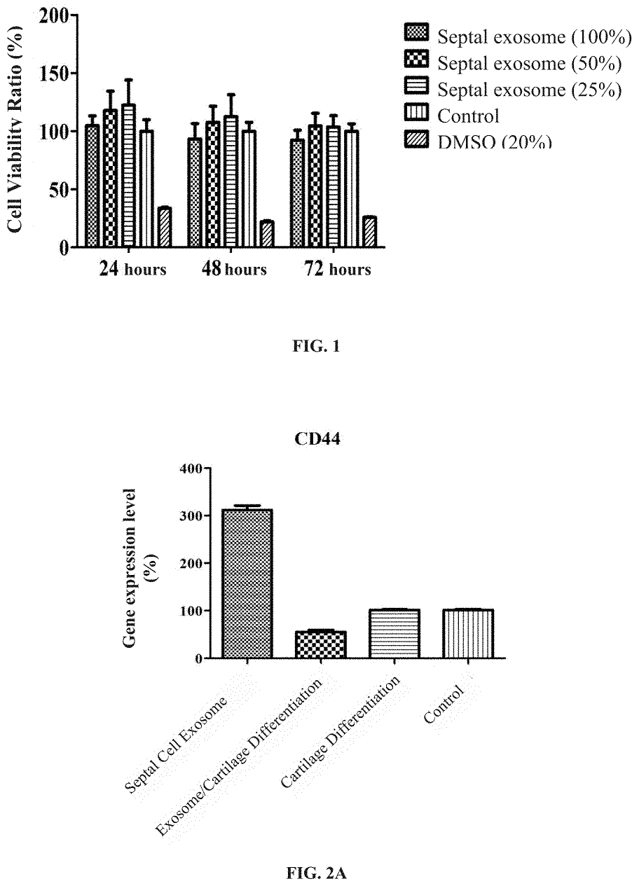

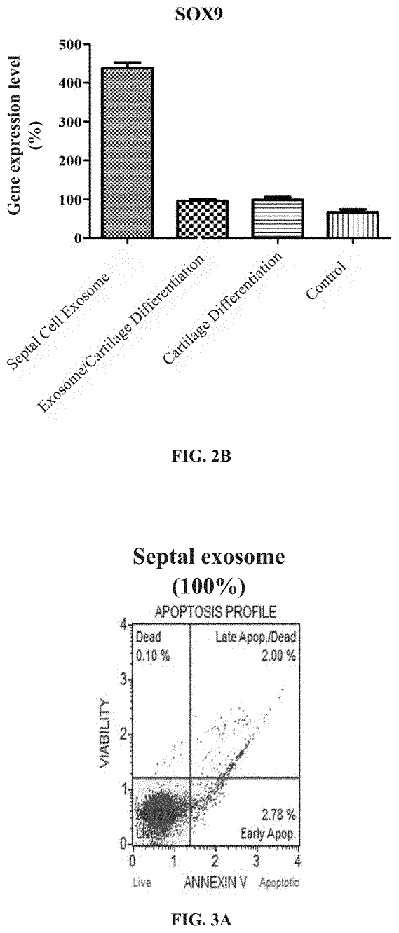

### DETAILED DESCRIPTION OF THE EMBODIMENTS

The present invention relates to developing a formulation produced by the microvesicles that are released by the cells isolated from septal cartilage for generation of cartilage tissue. In the implementation of the invention, septal cartilage stem cells are used. It is observed that the microvesicles obtained from cartilage cells have an effect on cartilage differentiation of stem cells. The effective range of these microvesicles is determined as 5-100% by volume. The microvesicles can be dissolved with a solution of dH2O, EtOH, cell culture medium, PBS, DMSO and mixtures thereof. The isolation of these exosomes from a cartilage-derived cell has provided these exosomes the ability to form cartilage, which likewise incorporates the inflammation suppression property of the stem cells. Therefore, the fact that these exosomes both enhance cartilage formation and suppress inflammation has been experimentally proven and shown in the figures. Because of these properties, these exosomes can be used in the treatment of cartilage damage and immune system-related diseases.

One of the differences of the formulation of the present invention with respect to the state of the art is the use of cells isolated from septal cartilage and it makes a significant difference both in terms of where it is isolated from and in use of characteristically different cell types. Furthermore, within the scope of the invention, exosomes which are a special component of these cells are used. These exosomes are only a part of the chemicals that are released by the cells outside of the cell.

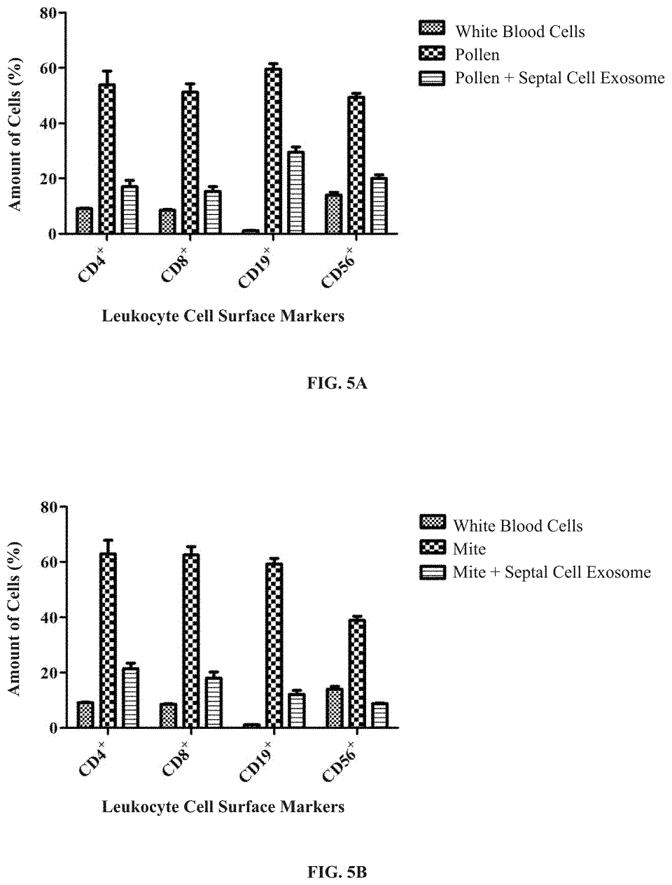

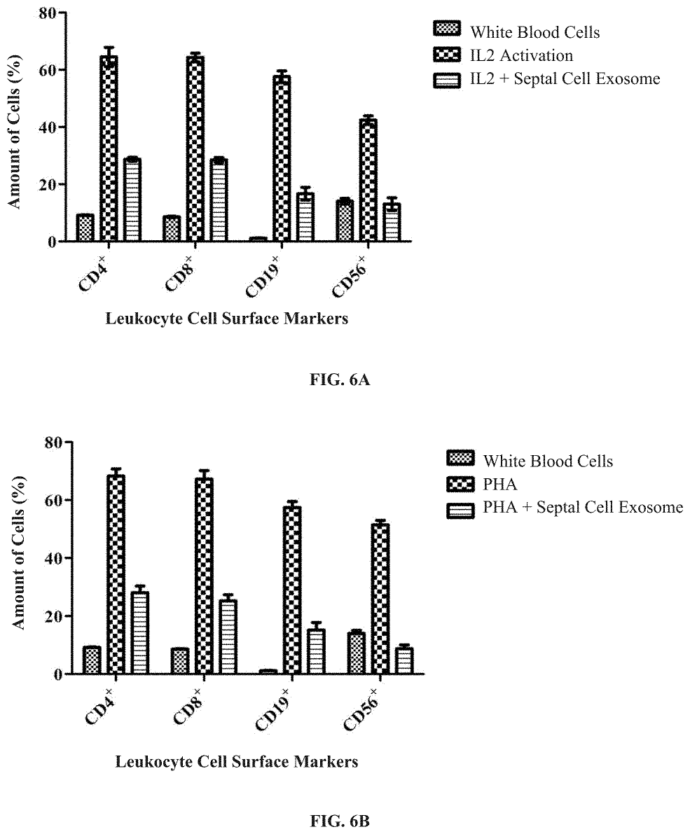

Within the scope of the invention, the use of the exosomes of septal cells increases cartilage tissue formation and does not cause any inflammation even though it is not autologous. These stem cell-derived exosomes, which have the immune suppressant property of the stem cells, do not cause an inflammation although they are not autologous and also suppress the occurring inflammation (FIGS. 5A, 5B, 6A and 6B). Within the scope of the invention, the exosomes released to the medium by the undifferentiated septal cells, which are not exposed to any chemicals, are isolated.

Isolated septal cartilage exosomes induce formation of cartilage tissue for use in the treatment of cartilage tissue defects such as osteoarthritis, costochondral joint inflammation, Tietze syndrome or arthrosis; and thanks to their anti-inflammatory properties, they enable formation of cartilage which does not produce immune response, inflammation, toxicity and irritation to the body and the cells. The method of forming cartilage tissue from these isolated septal cartilage exosomes within the scope of the invention comprises the steps of
culturing the cartilage cells in Dulbecco's modified Eagle's medium (DMEM) containing 10% exosome-depleted fetal bovine serum (Invitrogen) and 1% PSA (Biological Industries, Beit Haemek, Israel) in cell culture incubators at a temperature of 37° C. with 5% CO₂,
using exosome isolation solution containing biphasic PEG-Dextran for microvesicle isolation from the cells in the cultured medium,
centrifuging the medium collected from the culture medium at 300 g for 10 minutes in order to remove the waste cells,
transferring the supernatant to a new tube and centrifuging at 14000 g for 30 minutes in order to remove possible cell components,
transferring the supernatant to a new tube, adding 1/1 volume of PEG-Dextran solution thereon, centrifuging at 1000 g for 10 minutes, and then collecting the exosomes remaining in the lower phase,
administering a differentiation solution for cartilage differentiation to the septal cartilage exosome every other day for a period of 10 days,
obtaining the cartilage tissue as a result of differentiation.

Experimental Studies

3. Toxicity

After the cells were seeded in 96-well culture plates (Corning Glasswork, Corning, N.Y.) at 5000 cells/well in Dulbecco's modified Eagle's medium (DMEM) containing 10% exosome-depleted fetal bovine serum (Invitrogen) and 1% PSA (Biological Industries, Beit Haemek, Israel) in the culture medium, the viability levels of the cells were measured on day 1, 2 and 3. Cell viability was determined by using 3-(4,5-di-methyl-thiazol-2-yl)-5-(3-carboxy-methoxy-phenyl)-2-(4-sulfo-phenyl)-2H-tetrazolium (MTS)-method (CellTiter96 AqueousOne Solution; Promega, Southampton, UK). 10 μl MTS solution was added onto the cells within a 100 μl medium and it was incubated at 37° C. in dark for 2 hours. After the incubation process, cell viability was observed by performing absorbance measurement via ELISA plate reader (Biotek, Winooski, Vt.) device at 490 nm wavelength.

4. Cartilage Differentiation

The cells were seeded in 6-well culture plates (Corning Glasswork, Corning, N.Y.) at 50,000 cells/well in Dulbecco's modified Eagle's medium (DMEM) containing 10% exosome-depleted fetal bovine serum (Invitrogen) and 1% PSA (Biological Industries, Beit Haemek, Israel) in the culture medium. The following day the septal cartilage exosome and the differentiation solution used for cartilage differentiation in the literature were administered for 10 days every other day.

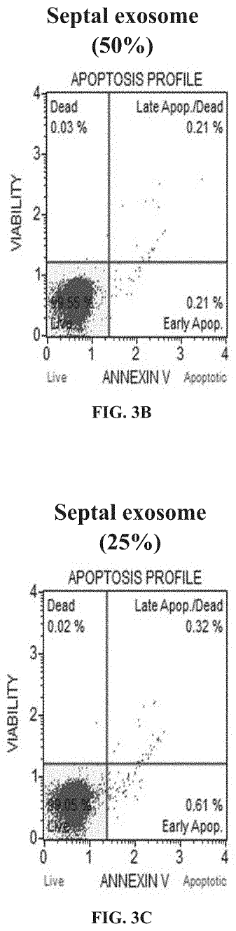

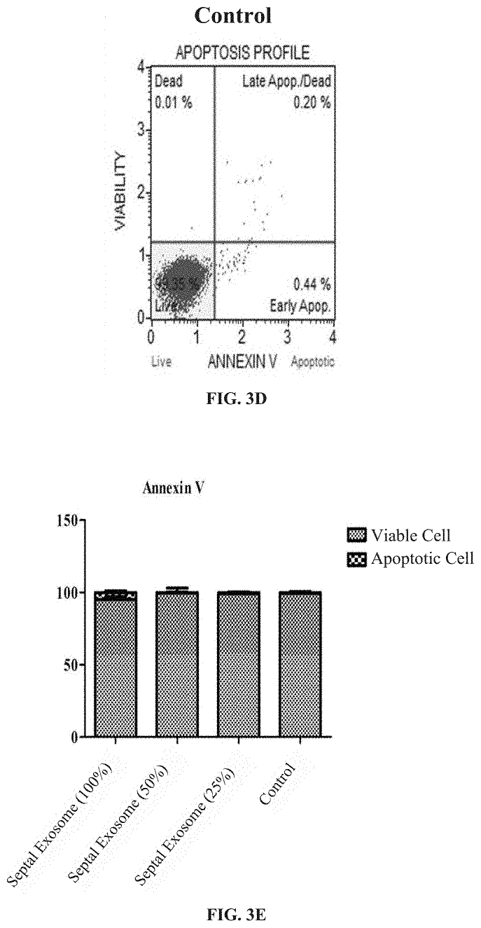

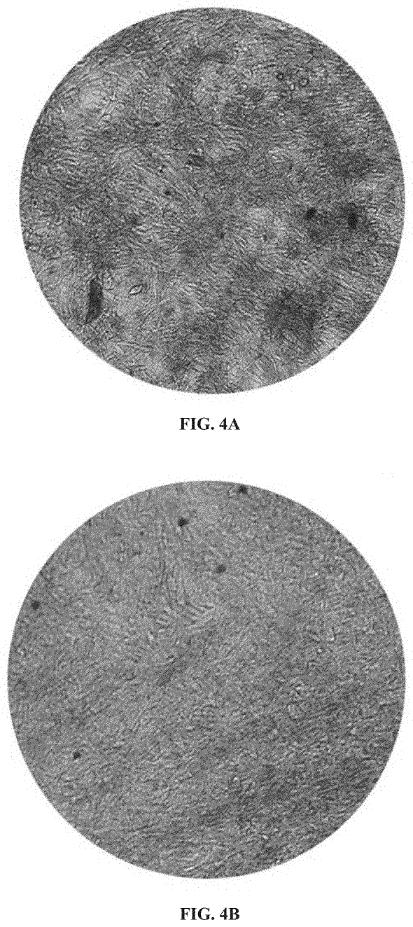

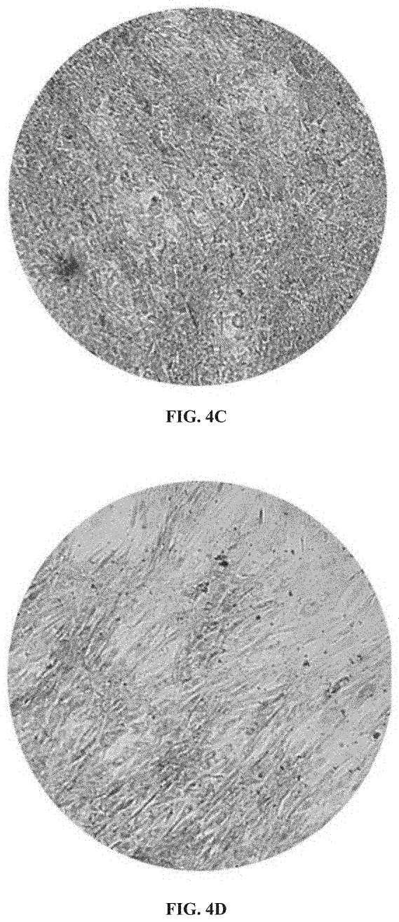

The effects of the medium used in cartilage differentiation and the exosomes obtained from septal cells on cartilage differentiation were compared, and the exosomes were shown to be more effective (FIGS. 3A-3E, 4A-4D, 5A and 5B).

Real Time PCR

Cultured cells may lose their own properties and acquire new properties. These properties may be both in morphological level and gene expression level. Real Time PCR method was applied to observe the changes in gene expression level. Total RNAs were isolated and cDNA was synthesized from the cells that were seeded in 6-well culture plates (Corning Glasswork, Corning, N.Y.) at 50,000 cells/well in Dulbecco's modified Eagle's medium (DMEM). The synthesized cDNAs were mixed with primers in Fermentas Maxima SYBR Green mixture product such that the final volume will be 20 μl and the expression levels of the genes were analyzed by using BIO-RAD device.

The advantages of the method of the present invention for generation of cartilage tissue from the isolated septal cartilage exosomes can be listed as follows:
Induces cartilage formation.
Has an inflammation suppressant property.
Does not cause inflammation.
Does not induce toxicity in cells.
Can be metabolized in the cell after use.
Can be used in the treatment of osteoarthritis and arthrosis.
Can be used in the treatment of cartilage tissue defects.
Can be used in nasal reconstruction.
Has a high potential of inducing cartilage formation for aesthetic and therapeutic purposes.
Can be used as an effective agent in tissue engineering.
Does not induce immune response against the body and the cells, inflammation, toxicity and irritation thanks to its anti-inflammatory properties.
Can be used in auto-immune diseases thanks to its immunosuppressant activity.
Can be used in treatment of rheumatoid arthritis thanks to its properties of enabling cartilage formation and suppressing inflammatory response.

### REFERENCES

[1]. Deans, R. J., & Moseley, A. B. (2000). Mesenchymal stem cells: biology and potential clinical uses. Experimental hematology, 28(8), 875-884.
[2]. Doral, M. N., Dönmez, G., Atay, Ö. A., Bozkurt, M., Leblebicio\check{g}lu, G., Üzumcügil, A., & Aydo\check{g}, T. 2007. “Dejeneratif eklem hastahklan”, TOTBİD dergisi, 6, 56-65.
[3]. Da\check{g}li, A. Ş., Özdem, C., Akalin, Y., Ensari, S. 1993. “Rinoplastide Biyomateryeller”, K.B.B. ve Baş Boyun Cerrahisi Dergisi, Cilt: 1 Sayi: 2.
[4]. Rotter, N., Bonassar, L. J., Tobias, G., Lebl, M., Roy, A. K., Vacanti, C. A. 2002. “Age dependence of biochemical and biomechanical properties of tissue-engineered human septal cartilage”, Biomaterials, 23(15), 3087-3094.
[5]. Şenköylü, A., & Korkusuz, F. 2004. “Kikirdak Onariminda Doku Mühendisli\check{g}i Uygulamalan”, TOTBİD (Türk Ortopedi ve Travmatoloji Birli\check{g}i Derne\check{g}i) Dergisi, 3, 3-4.
[6]. Smith, G. D., Knutsen, G., & Richardson, J. B. 2005. “A clinical review of cartilage repair techniques”, Bone & Joint Journal, 87(4), 445-449.
[7]. Haisch, A., Duda, G. N., Schroeder, D., Gröger, A., Gebert, C., Leder, K., & Sittinger, M. 2005. “The morphology and biomechanical characteristics of subcutaneously implanted tissue-engineered human septal cartilage”, European Archives of Oto-Rhino-Laryngology and Head &Neck, 262(12), 993-997.
[8]. Li, M. H., Xiao, R., Li, J. B., & Zhu, Q. 2017. “Regenerative approaches for cartilage repair in the treatment of osteoarthritis”, Osteoarthritis and cartilage, 25(10), 1577-1587.

1. Isolated septal cartilage exosome used for inducing cartilage tissue formation due to the fact that stem cells are effective on cartilage differentiation.

2. Isolated septal cartilage exosome according to claim 1, which is effective when used 5-100%.

3. Isolated septal cartilage exosome according to claim 1, which can be dissolved with a solution selected from a group consisting of dH2O, EtOH, cell culture medium, PBS, DMSO and mixtures thereof.

4. Isolated septal cartilage exosome according to claim 1, which is used for treatment of cartilage tissue defects such as osteoarthritis, costochondral joint inflammation, Tietze syndrome or arthrosis.

5. Method of generating cartilage tissue from the isolated septal cartilage exosomes according to claim 1, comprising the steps of
culturing the cartilage cells in Dulbecco's modified Eagle's medium (DMEM) containing 10% exosome-depleted fetal bovine serum (Invitrogen) and 1% PSA (Biological Industries, Beit Haemek, Israel) in cell culture incubators,
using exosome isolation solution containing biphasic PEG-Dextran for microvesicle isolation from the septal cartilage cells in the cultured medium,
centrifuging the medium collected from the culture medium at 300 g for 10 minutes in order to remove the waste cells,
transferring the supernatant to a new tube and centrifuging at 14000 g for 30 minutes in order to remove possible cell components,
transferring the supernatant to a new tube, adding 1/1 volume of PEG-Dextran solution thereon, centrifuging at 1000 g for 10 minutes, and then collecting the exosomes remaining in the lower phase,
administering a differentiation solution for cartilage differentiation to the septal cartilage exosome every other day for a period of 10 days,
obtaining the cartilage tissue as a result of differentiation.

6. Method of generating cartilage tissue from the isolated septal cartilage exosomes according to claim 3, wherein the cartilage cells are cultured in cell culture incubators at a temperature of 37° C. with 5% CO₂.
